(19) United States
(12) Patent Application Publication
(10) Pub. No.: US 2015/0140125 A1
(43) Pub. Date: May 21, 2015

(54) SYNERGISTIC CANCER THERAPY DRUG COMBINATIONS

(71) Applicant: Life Plus, LLC, Sterling, KS (US)

(72) Inventors: Gene H. Zaid, Sterling, KS (US); Thomas W. Burgoyne, Sterling, KS (US); Lisa Stehno-Bittel, Bonner Springs, KS (US); Mary Ann Lee-Stanislav, Kansas City, MO (US)

(21) Appl. No.: 14/543,832

(22) Filed: Nov. 17, 2014

Related U.S. Application Data
(60) Provisional application No. 61/906,183, filed on Nov. 19, 2013.

(51) Int. Cl. A61K 36/888; A61K 31/337; A61K 31/513; A61K 33/24; A61K 38/08; A61K 31/202; A61K 31/519; A61K 31/196; A61K 9/10; A61K 9/14; A61K 31/575; A61K 31/11; A61K 31/704; A61K 31/439

(52) U.S. Cl. CPC A61K 36/888; A61K 31/704; A61K 31/337; A61K 31/513; A61K 33/24; A61K 38/08; A61K 31/439; A61K 31/519; A61K 31/196; A61K 9/10; A61K 9/14; A61K 31/575; A61K 31/11; A61K 31/202
USPC 424/649; 514/34; 514/19.3; 424/725; 514/171

(57) ABSTRACT

Synergistic cancer therapy drug combinations include therapeutically effective amounts of at least one chemotherapeutic drug or agent with a fortified decoction dosage form comprising from about 10 mg to about 6,000 mg each of β-sitosterol, isovanillin, and linolenic acid. The decoction dosage preferably includes plant extract(s) of the genus Arum fortified with effective amounts of β-sitosterol, isovanillin, and linolenic acid not derived from the plant. The combination may be in various forms including aqueous dispersions, gels, ampules, powders, capsules, pills, or tablets, and are normally administered orally to patients. The anticancer combinations have therapeutic effects on cancerous tissue which are greater than the sum of the individual therapeutic effects of the fortified decoction dosage form and the at least one chemotherapeutic agent on the cancerous tissue.

### CROSS-REFERENCE TO RELATED APPLICATION

This application claims the benefit of identically titled application Ser. No. 61/906,183, filed Nov. 19, 2013. This earlier application is incorporated by reference herein in its entirety.

### BACKGROUND OF THE INVENTION

1. Field of the Invention

The present invention relates to synergistic combination therapeutics for treating cancerous tissue and methods for the treatment of human cancers, including daily dosage forms for administration to cancer patients, and methods of formulating and administering such dosage forms in combination with one or more chemotherapeutic agent(s) to yield synergistic improvements in treatment outcomes. More particularly, the invention is concerned with the administration of chemotherapeutics in synergistic combination with daily dosage forms (e.g., aqueous mixtures, capsules, pills, or tablets) of Arum extract and further containing from about 10 mg to about 6,000 mg of each of β-sitosterol, isovanillin, and linolenic acid. Such treatment provides a marked decline and/or elimination of cancerous tissues and cells, and a corresponding enhancement of the wellness and lifestyles of the treated patients.

2. Description of Related Art

Cancer is a generic term for a large group of diseases that can affect any part of the body. Other terms used are malignant tumors and neoplasms. One defining feature of cancer is the rapid creation of abnormal cells that grow beyond their usual boundaries, and which can then invade adjoining parts of the body and spread to other organs. This process is referred to as metastasis. Metastases are the major cause of death from cancer.

The transformation from a normal cell into a tumor cell is a multistage process, typically a progression from a pre-cancerous lesion to malignant tumors. These changes are the result of the interaction between a person's genetic factors and three categories of external agents, including:
physical carcinogens, such as ultraviolet and ionizing radiation
chemical carcinogens, such as asbestos, components of tobacco smoke, aflatoxin (a food contaminant) and arsenic (a drinking water contaminant)
biological carcinogens, such as infections from certain viruses, bacteria or parasites.

Some examples of infections associated with certain cancers:
Viruses: hepatitis B and liver cancer, Human Papilloma Virus (HPV) and cervical cancer, and human immunodeficiency virus (HIV) and Kaposi sarcoma.
Bacteria: Helicobacter pylori and stomach cancer.
Parasites: schistosomiasis and bladder cancer.

Aging is another fundamental factor for the development of cancer. The incidence of cancer rises dramatically with age, most likely due to a buildup of risks for specific cancers that increase with age. The overall risk accumulation is combined with the tendency for cellular repair mechanisms to be less effective as a person grows older. Tobacco use, alcohol use, low fruit and vegetable intake, and chronic infections from hepatitis B (HBV), hepatitis C virus (HCV) and some types of Human Papilloma Virus (HPV) are leading risk factors for cancer in low- and middle-income countries. Cervical cancer, which is caused by HPV, is a leading cause of cancer death among women in low-income countries. In high-income countries, tobacco use, alcohol use, and being overweight or obese are major risk factors for cancer.

The most common cancer treatment modalities are surgery, chemotherapy, and radiation treatments. All of these techniques have significant drawbacks in terms of side effects and patient discomfort. For example, chemotherapy may result in significant decreases in white blood cell count (neutropenia), red blood cell count (anemia), and platelet count (thrombocytopenia). This can result in pain, diarrhea, constipation, mouth sores, hair loss, nausea, and vomiting.

Biological therapy (sometimes called immunotherapy, biotherapy, or biological response modifier therapy) is a relatively new addition to the family of cancer treatments. Biological therapies use the body's immune system, either directly or indirectly, to fight cancer or to lessen the side effects that may be caused by some cancer treatments.

Despite the immense amount of worldwide research and efforts to stem the tide of cancer and its side effects, the disease in its many manifestations continues to be a huge problem. Therefore, any new cancer treatment having a curative affect and/or the ability to ameliorate cancer symptoms and improve the lifestyle of patients is highly significant and important.

### SUMMARY

In one or more embodiments, a therapeutic anticancer combination is provided. The therapeutic combination comprises (or consists essentially or even consists of) coordinated, preselected therapeutically-effective amounts of at least one chemotherapeutic agent and a fortified decoction dosage form, wherein the fortified decoction dosage form comprises from about 10 mg to about 6,000 mg of β-sitosterol, isovanillin, and linolenic acid (more preferably from about 1,000-4,000 mg each, still more preferably from about 2,500-3,500 mg each, and most preferably about 3,000 mg each). The combinations have therapeutic effects on cancerous tissue which are greater than the sum of the individual therapeutic effects of the fortified decoction dosage form and the at least one chemotherapeutic agent on the cancerous tissue. In one aspect, the fortified decoction dosage form comprises (or consists essentially or even consists of) plant extract of the genus Arum, fortified with effective amounts of β-sitosterol (phytosterol), isovanillin (phenolic aldehyde), and linolenic acid (fatty acid) not derived from the plant extract. The extract and β-sitosterol, isovanillin, and linolenic acid can optionally be dispersed or dissolved in a solvent system (e.g., in suspension or solution).

Methods of treating human cancers are also described herein. The methods generally comprise (or consist essentially or even consist of) administering a fortified decoction dosage form to a subject in need thereof, in combination with a chemotherapeutic agent. The fortified decoction dosage form can be administered on a daily basis. The chemotherapeutic agent can be administered as part of the method. Alternatively, the subject may already be undergoing chemotherapy treatment with the chemotherapeutic agent, in which case, the fortified decoction dosage form is added as an adjunctive therapy to the chemotherapeutic agent (or vice versa). As demonstrated in the Examples, the fortified decoction dosage form in combination with said chemotherapeutic agent advantageously achieves a therapeutic synergy in treating the cancer in the subject. As such, the cancer of the subject is improved as compared to a subject treated with a chemotherapeutic agent not in combination with the fortified decoction dosage form. The respective amounts of the fortified decoction and chemotherapy are coordinated with each other so as to obtain the desired therapeutic synergy, i.e., preselected amounts are of each are employed to achieve the ends of the invention.

### BRIEF DESCRIPTION OF THE DRAWINGS

FIG. 1 shows the chemical structure of doxorubicin;

FIG. 2 is a pair of dose/response curves for GZ17 alone (left-hand graph) and doxorubicin alone (right-hand graph) on lung cancer cells from Example 1;

FIG. 3 is a dose/response curve indicating synergy of GZ17 and the chemotherapeutic doxorubicin beyond their individual additive effects, on lung cancer cells;

FIG. 4 shows the chemical structure of paclitaxel;

FIG. 5 is a pair of dose/response curves for GZ17 alone (left-hand graph) and paclitaxel alone (right-hand graph) on lung cancer cells from Example 2;

FIG. 6 is a graph showing the combined effects of GZ17 and paclitaxel on lung cancer cells;

FIG. 7 shows the chemical structure of fluorouracil;

FIG. 8 is a pair of dose/response curves for GZ17 alone (left-hand graph) and fluorouracil alone (right-hand graph) on lung cancer cells from Example 2;

FIG. 9 is a dose/response curve indicating synergy of GZ17 and the chemotherapeutic fluorouracil beyond their individual additive effects, on lung cancer cells;

FIG. 10 is a pair of dose/response curves for GZ17 alone (left-hand graph) and doxorubicin alone (right-hand graph) on ovarian cancer cells from Example 3;

FIG. 11 is a graph indicating synergy of GZ17 and the chemotherapeutic doxorubicin beyond their individual additive effects, on ovarian cancer cells;

FIG. 12 is a pair of dose/response curves for GZ17 alone (left-hand graph) and paclitaxel alone (right-hand graph) on ovarian cancer cells from Example 3;

FIG. 13 is a graph indicating synergy of GZ17 and the chemotherapeutic paclitaxel beyond their individual additive effects, on ovarian cancer cells;

FIG. 14 is a pair of dose/response curves for GZ17 alone (left-hand graph) and fluorouracil alone (right-hand graph) on ovarian cancer cells from Example 3;

FIG. 15 is a graph indicating synergy of GZ17 and the chemotherapeutic fluorouracil beyond their individual additive effects, on ovarian cancer cells;

FIG. 16 shows the chemical structure of cisplatin;

FIG. 17 is a graph indicating synergy of GZ17 and the chemotherapeutic cisplatin on ovarian cancer cells from Example 3;

FIG. 18 is an 8×8 graph indicating synergy of GZ17 and the chemotherapeutic doxorubicin beyond their individual additive effects, using eight different doses of GZ17 and eight different doses of doxorubicin, resulting in 64 different dose combinations tested in 6 replicates per dose, from Example 4;

FIG. 19 is a graph indicating synergy of GZ17 and the chemotherapeutic actinomycin D beyond their individual additive effects, on ovarian cancer cells, from Example 4;

FIG. 20 is a graph indicating synergy of GZ17 and the chemotherapeutic vinblastine beyond their individual additive effects, on lung cancer cells, from Example 4;

FIG. 21 is a graph indicating synergy of GZ17 and the chemotherapeutic vinblastine beyond their individual additive effects, on ovarian cancer cells, from Example 4;

FIG. 22 is a graph indicating synergy of GZ17 and the chemotherapeutic methotrexate beyond their individual additive effects, on lung cancer cells, from Example 4;

FIG. 23 is a graph indicating synergy of GZ17 and the chemotherapeutic methotrexate beyond their individual additive effects, on ovarian cancer cells, from Example 4;

FIG. 24 is a graph indicating synergy of GZ17 and the chemotherapeutic chlorambucil beyond their individual additive effects, on lung cancer cells, from Example 4; and

FIG. 25 is a graph indicating synergy of GZ17 and the chemotherapeutic chlorambucil beyond their individual additive effects, on ovarian cancer cells, from Example 4.

### DETAILED DESCRIPTION

The present invention is concerned with therapeutic anticancer combinations having a synergistic effect in the treatment of cancers. The combinations generally comprise a dosage form of a fortified decoction containing Arum extract and from about 10 mg to about 6,000 mg (more preferably from about 1,000-4,000 mg, still more preferably from about 2,500-3,500 mg, and most preferably about 3,000 mg) of each of β-sitosterol, isovanillin, and linolenic acid in combination with a chemotherapeutic agent. The therapeutic effectiveness of the combination is greater than that of the dosage form alone or the administration of one or more of the chemotherapeutic drug(s) without the dosage form.

The fortified decoction can be provided as an aqueous dispersion dosage form or in an alternative dosage form derived from the fortified decoction, such as a gel, ampule, powder, capsule, pill, and/or tablet. Suitable fortified decoctions and methods of making the same are described in U.S. Pat. No. 8,039,025, incorporated by reference herein in its entirety. The dosage form can be prepared employing a decoction or tea using plant parts (preferably leaves and/or roots, and most preferably roots) of Arum palaestinum Boiss, or any other suitable plant parts of the genus Arum. The plant decoction (Arum extract) is then supplemented or fortified using effective amounts of β-sitosterol, isovanillin, and linolenic acid, which are not derived from the plant decoctions. Advantageously, amounts of (preferably essentially pure) β-sitosterol, isovanillin, and linolenic acid are added to the plant decoctions to achieve the foregoing amounts of these ingredients. The linolenic acid may be added in the acid form or as a salt (e.g., sodium or potassium salt). Thus, the fortified decoction dosage form generally comprises plant extract of the genus Arum, which is fortified with effective amounts of β-sitosterol, isovanillin, and linolenic acid to achieve the desired ranges indicated above.

In one or more embodiments, the fortified decoction dosage form consists essentially of plant extract of the genus Arum, fortified with effective amounts of β-sitosterol, isovanillin, and linolenic acid, optionally dispersed or dissolved in an aqueous solvent system (i.e., in solution). In one or more embodiments, the decoction (or fortified decoction) can be (freeze-dried) to obtain a dried extract for use in powder, capsules, tablets, and other solid dosage forms. Dried extracts and/or fortified extracts can also be formed by evaporation (e.g., rotovap), heating, vacuum, distillation, filtering, and combinations thereof. In or more embodiments, the fortified decoction solution is filtered one or more times through filter paper to capture solids that can be dried. The liquid portion can also be dried as described above to isolate any remaining solids from the decoction or fortified decoction solution. The solids can then be combined to create powder, capsule, pill, and/or tablet dosage forms. The solids can also be re-dispersed in a suitable pharmaceutically-acceptable carrier, diluent, or vehicle for dosing. Thus, the fortified decoction can also be used in liquid or gel dosage form. References herein to the “fortified decoction dosage form” encompass all liquid and non-liquid dosage forms of the formulation, including solids and gels.

The fortified decoction dosage form is used in synergistic combination with a chemotherapeutic agent. A “chemotherapeutic” or “chemotherapeutic agent” refers to a chemical compound useful in the treatment of cancer. Chemotherapeutics may be selectively toxic or destructive of cancerous tissue and/or cells, but also includes indiscriminately cytotoxic compounds used in cancer treatments. Exemplary chemotherapeutics include antimetabolites, anthracyclines, mitotic inhibitors, alkylating agents, and combinations thereof.

Additional ingredients may be included either with the fortified decoction dosage form and/or the chemotherapeutic agent(s) for administration to the subject. Such additional ingredients include, other active agents, preservatives, buffering agents, salts, carriers, diluents, or other pharmaceutically-acceptable ingredients. The active agents that could be included in the compositions include antiviral, antibiotic, or other anticancer compounds.

The therapeutic combination will comprise a therapeutically effective amount of the fortified decoction dosage form and a therapeutically effective amount of the chemotherapeutic agent(s). As used herein, a “therapeutically effective” amount refers to the amount that will elicit the biological or medical response of a tissue, system, or subject that is being sought by a researcher or clinician, and in particular elicit some desired therapeutic effect as against the cancerous tissue by preventing and/or inhibiting proliferation and/or survival of cancerous cells, and/or slowing the progression of the cancer. One of skill in the art recognizes that an amount may be considered therapeutically “effective” even if the condition is not totally eradicated or prevented, but it or its symptoms and/or effects are improved or alleviated partially in the subject. The Physician's Desk Reference (PDR) discloses dosages of various known chemotherapeutic agents. The respective dosing regimens and dosages which are therapeutically effective will depend on the particular cancer being treated, the extent of the disease, and other factors related to the patient as determined by those of ordinary skill in the art.

The terms “therapeutic” or “treat,” as used herein, refer to products or processes that are intended to produce a beneficial change in an existing condition (e.g., cancerous tissue, tumor size, metastases, etc.) of a subject, such as by reducing the severity of the clinical symptoms and/or effects of the cancer, and/or reducing the duration of the symptoms/effects of a subject.

In use, a therapeutically-effective amount of the therapeutic combination is administered to a subject in need thereof In some embodiments, a composition comprising a therapeutically-effective amount of the therapeutic combination (i.e., as a single unit dosage) is administered to a subject. In some embodiments, a therapeutically effective amount of the fortified decoction dosage form and a therapeutically effective amount of the chemotherapeutic agent(s) are co-administered to the subject in need thereof. The phrase “co-administer” is intended to embrace administration of each agent in a sequential manner as well as co-administration of these agents in a substantially simultaneous manner in doses given separately. Although described herein with respect to therapeutic treatments, the methods can be also applied for clinical research and/or study.

Advantageously, administration of the fortified decoction dosage form in combination with a chemotherapeutic agent(s) achieves an unexpected “therapeutic synergy.” This means that the synergistic anticancer combination has a therapeutic effect on the cancerous tissue which is greater than the sum of the individual therapeutic effects of the fortified decoction dosage form and chemotherapeutic agent(s) on the cancerous tissue. In other words, “therapeutic synergy” occurs when the treatment is improved over the treatment outcomes of either agent individually, and is greater than the expected sum of the individual therapeutic effects on the cancerous tissue.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### Example 1

The human alveolar adenocarcinoma cell line (H358) was used to create miniature 3-dimensional (3-D) tumors by loading the individual cells onto a micromold using techniques described in the publication (Ramachandran et al., 2013) and in U.S. Pat. App. Pub. No. 2010/0233239, incorporated by reference herein to the extent not inconsistent with the present disclosure. Cells were pipetted into the mold and allowed 3-5 days to form 3-D clusters, mimicking miniature tumors. The cell clusters were removed from the mold by washing and gentle pipetting. The cell clusters were placed in media containing 0.5% FBS. The clusters were dispensed into 96-well plates at a density of approximately 12-15 clusters per well for testing with the therapeutic composition designated herein as “GZ17” and a known chemotherapeutic agent (doxorubicin, aka adriamycin).

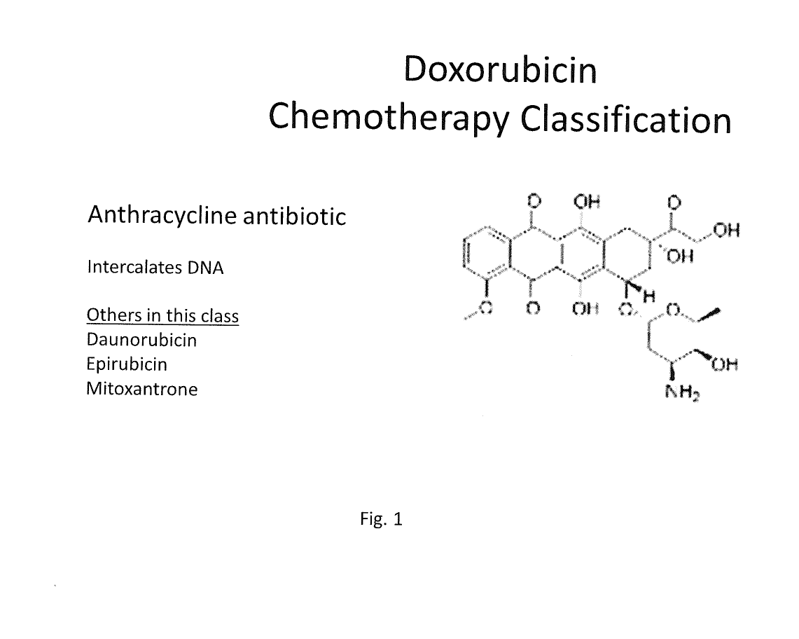

GZ17 is a composition comprising β-sitosterol, isovanillin, and linolenic acid in various dosage forms, including aqueous dispersions and powders, and is described in U.S. Pat. No. 8,039,025, incorporated by reference herein in its entirety. Doxorubicin is a member of the anthracycline antibiotic family of chemotherapeutics. It acts by intercalating DNA, so that DNA synthesis cannot occur. The tumor cells can't reproduce, and eventually they will die. The chemical structure is provided in FIG. 1. Other drugs in this class include daunorubicin, epirubicin, and mitoxantrone.

The cell clusters were tested against GZ17 and doxorubicin individually at various dosages, and then against the combination therapeutic containing both agents.

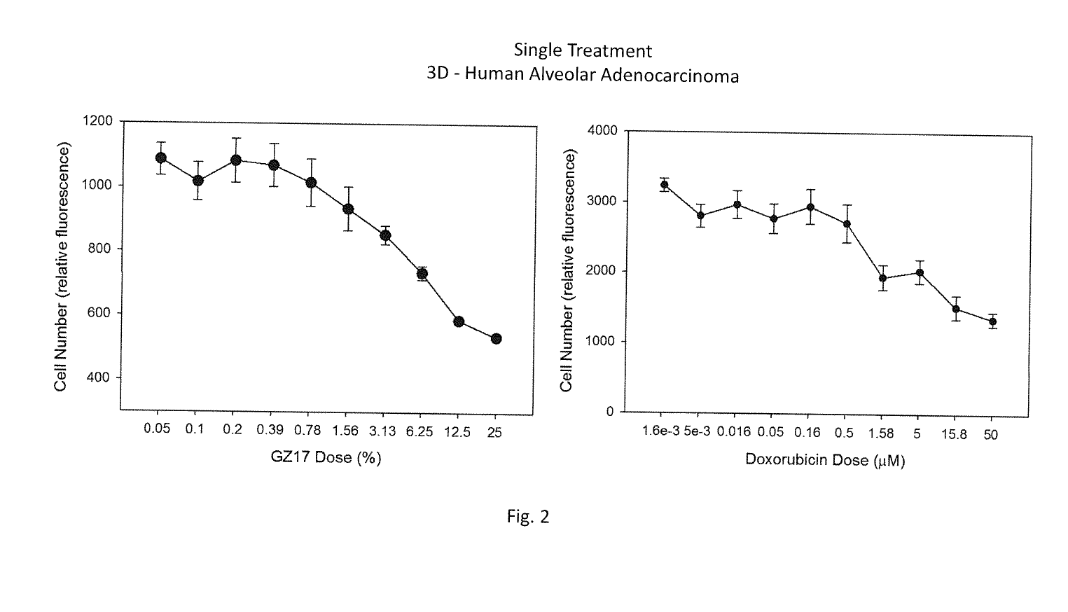

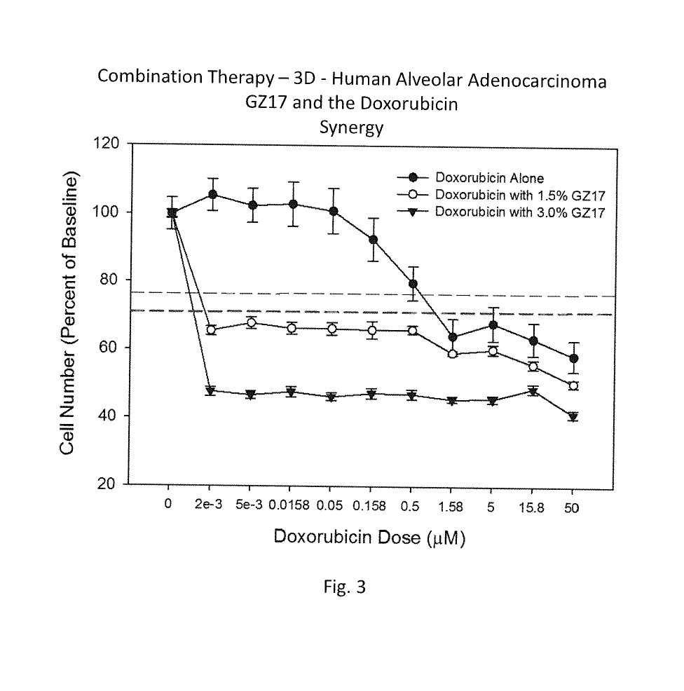

The results are shown in FIGS. 2-3. In FIG. 2, a standard dose/response curve is shown for GZ17 alone and doxorubicin alone. 96-well cell culture plates were loaded with increasing doses of GZ17 (graph on left) within a row. The doses were each added to 8 rows of each plate for 8 replicates at each dose. Cell clusters were pipetted into each well, and exposed for 24 hours to GZ17. At the completion of 24 hours, the wells were treated with 20 microliters of Prestoblue and allowed to incubate for up to 4 hours. The plates were read at 2-4 different time points after exposure to Prestoblue ranging from 30 minutes to 4 hours. The one-hour time point was the most often used and reported. The plates were loaded into a Perkin Elmer Enspire plate reader and each well was excited at 490 nm and read at 560 nm wavelengths. Relative fluorescence for each well was read.

FIG. 2 shows the mean±standard error for each dose of GZ17 tested. This Figure further shows the raw fluorescence values. As the concentration of GZ17 increased, the fluorescence declined, indicating a reduced number of live cells in the cell clusters.

Likewise, different cell clusters were exposed to increasing doses of doxorubicin rather than GZ17, using the same methods described above. Again, the fluorescence declined with increasing doses of doxorubicin.

FIG. 3 shows that while both GZ17 and doxorubicin reduced the cell number in each well, when combined, their effect was increased, indicating synergy of the GZ17 and the chemotherapy beyond their individual additive effects. The same methods described above were used for this experiment. Increasing doses of GZ17 were added to each row of a 96-well plate. This study was repeated 4 times, once with GZ17 alone, once with the increasing doses of GZ17 plus 0.16 μM doxorubicin, once with increasing doses of GZ17 plus 0.5 μM doxorubicin, and once with increasing doses of GZ17 plus 1.68 μM doxorubicin. A total of 6 replicates of each combination were performed. The upper dashed line in FIG. 3 indicates the decline in cell number (from 100%) for the same cells when exposed to 1.5% GZ17 alone (without doxorubicin). The lower dashed line indicates the percent of cell loss noted when the same cells were exposed to 3.0% GZ17 alone. Thus, any loss of cells beyond the upper line (for 1.5% GZ17) and lower line (for 3.0% GZ17) indicates a greater activity than either compound alone, thus defining synergy. At the lowest doses of doxorubicin (0.002, 0.005, 0.0158, 0.5 μM), the addition of GZ17 at both 1.5 and 3.0%, enhanced the effect of doxorubicin alone and was greater than the effect of GZ17 alone. Thus, at 0.002 doxorubicin, a statistically significant reduction in cell number was achieved with the lowest dose tested (1.5%) of GZ17. Comparing these results to the graph on the right of FIG. 2, one sees that 0.002 to 0.158 μM doxorubicin alone had no statistically significant reduction in the number of cells from the baseline. The data support the conclusion that there exists a synergy between the effects of GZ17 and doxorubicin, inducing cell death in human lung cancer.

In further detail, at the lowest dose of doxorubicin where the chemotherapy by itself had no effect, the combination of 1.5% GZ17 plus 0.002 □M doxorubicin had a 32% greater kill rate (percent dead cancer cells) than GZ17 alone. At a higher level of doxorubicin (1.58 μM) the doxorubicin alone caused 36% of the cancer cells to die, and 3% GZ17 alone caused 30% of the cancer cells to die, the two added together cause 56% of all cancer cells to die. This shows classic synergy between the two agents. If doxorubicin and GZ17 were acting through the same mechanism to cause cell death (i.e., binding to the same receptors on the cancer cells), then there could not be an additive effect.

### Example 2

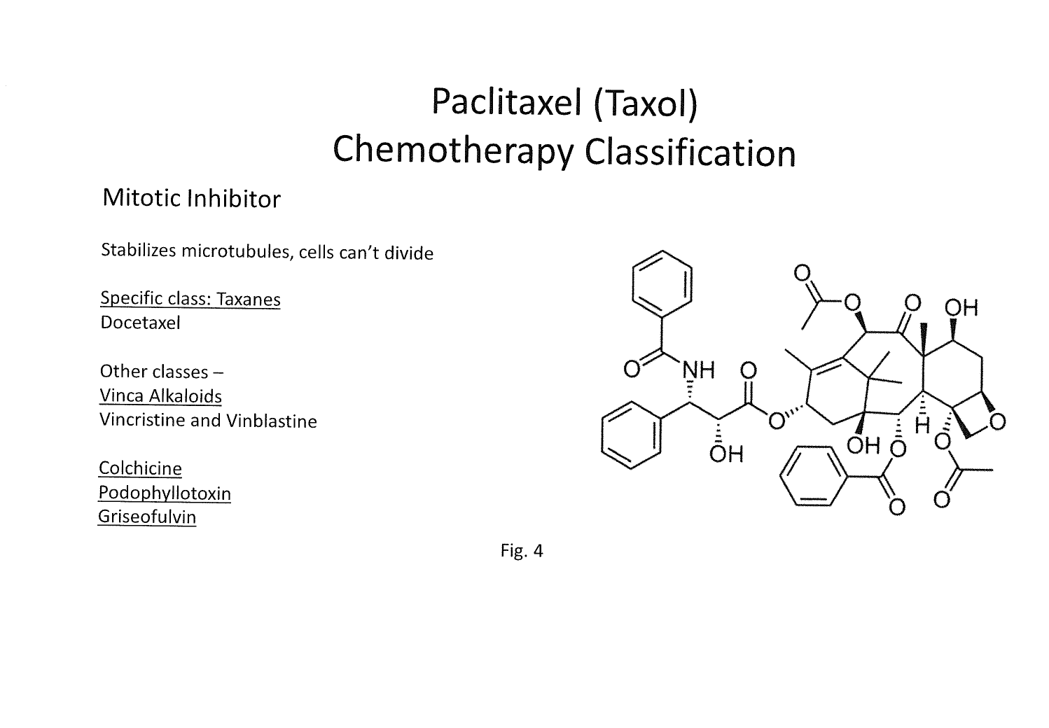

The same studies from Example 1 were repeated, but doxorubicin was replaced by paclitaxel. The chemotherapy classification of paclitaxel (also known as taxol) is a mitotic inhibitor. It works by stabilizing the microtubules. The microtubules are part of the cytoskeleton of the cell. When the cells go into cell division, the microtubules must disassemble so that the cell can divide. However, with paclitaxel, the microtubule polymer can't disassemble, and thus the cells can't divide which triggers apoptosis, or programmed cell death. The chemical structure of paclitaxel is shown in FIG. 4. There are subcategories of drugs in the mitotic inhibitor class including the vinca alkaloids (vincristine and vinblastine) and the colchicines class, podophyllotoxin and griseofulvin. Other drugs that work like paclitaxel are the taxanes and docotaxel.

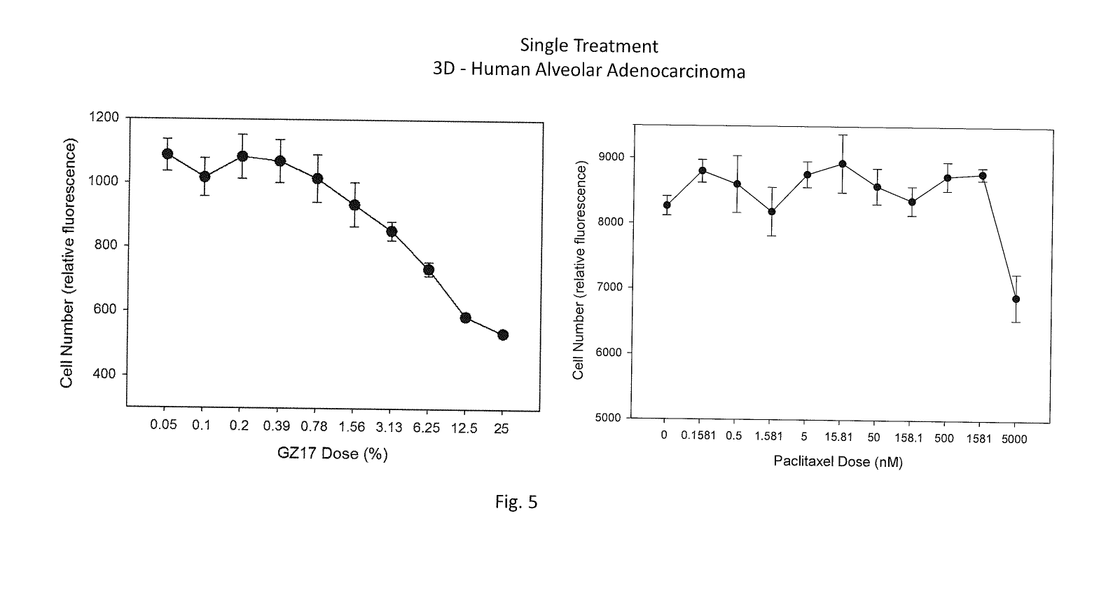

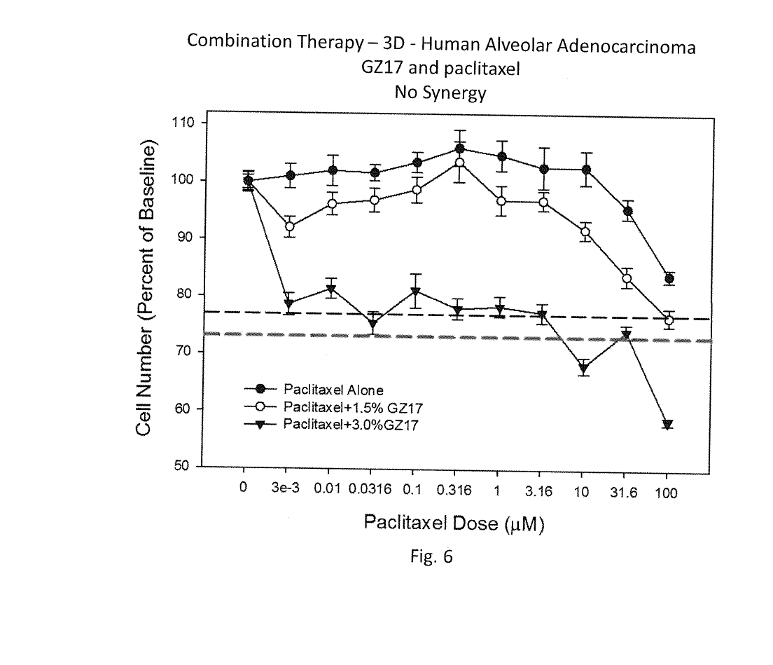

The results are shown in FIGS. 5-6. As shown in FIG. 5, with increasing doses of paclitaxel (graph on right), there is very little effect on the number of cells alive in each cluster until an extremely high dose of paclitaxel (5000 nM) is reached. All other doses of paclitaxel failed to reduce the number of live cells in the cluster to a statistically significant level.

FIG. 6 demonstrates that when GZ17 and paclitaxel were added together, only the effect of GZ17 is measured. When GZ17 and paclitaxel were added together, only the effect of GZ17 is measured, and if anything the paclitaxel reduced the effect of GZ17 alone. Neither the addition of 1.5 or 3.0% GZ17 to paclitaxel (open circles and black triangles, respectively) brought the cell death level to the level of GZ17 alone at those doses (upper dashed line=1.5% GZ17 and lower dashed line=3.0% GZ17). Thus, there is no additive effect of GZ17 to paclitaxel on these human alveolar adenocarcinoma cells. However, this data confirms that GZ17 alone has a therapeutic effect on cancerous tissue.

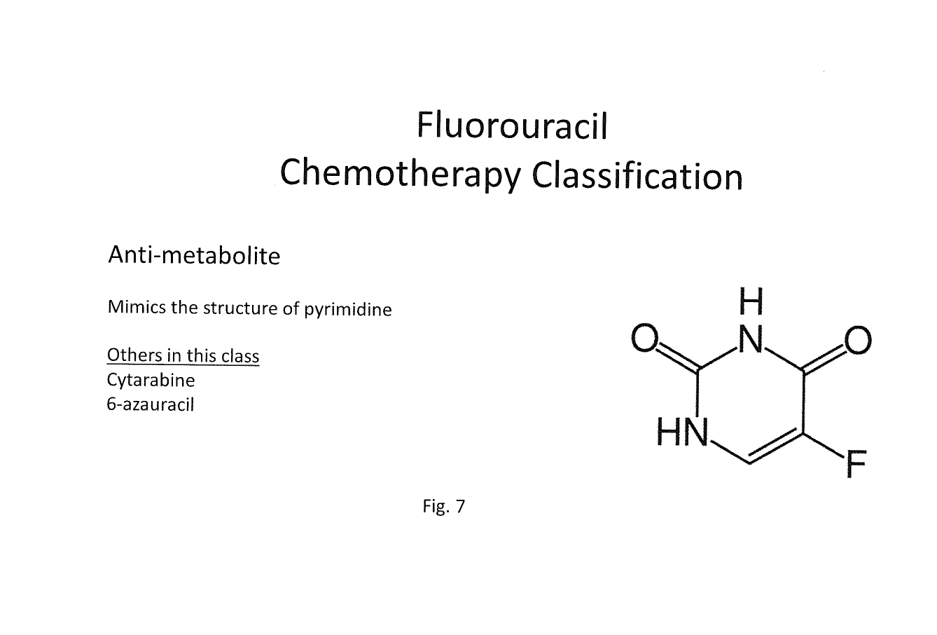

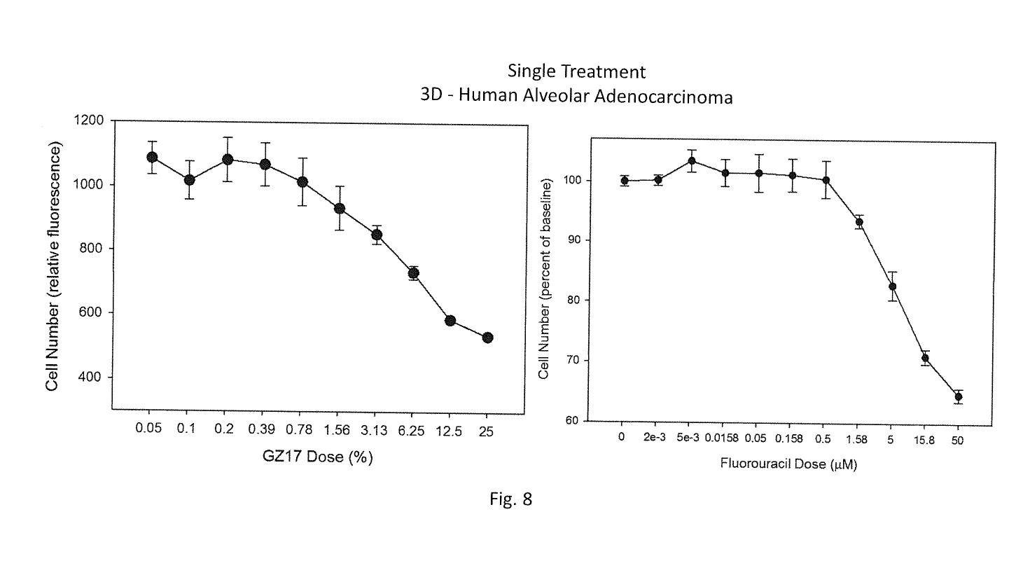

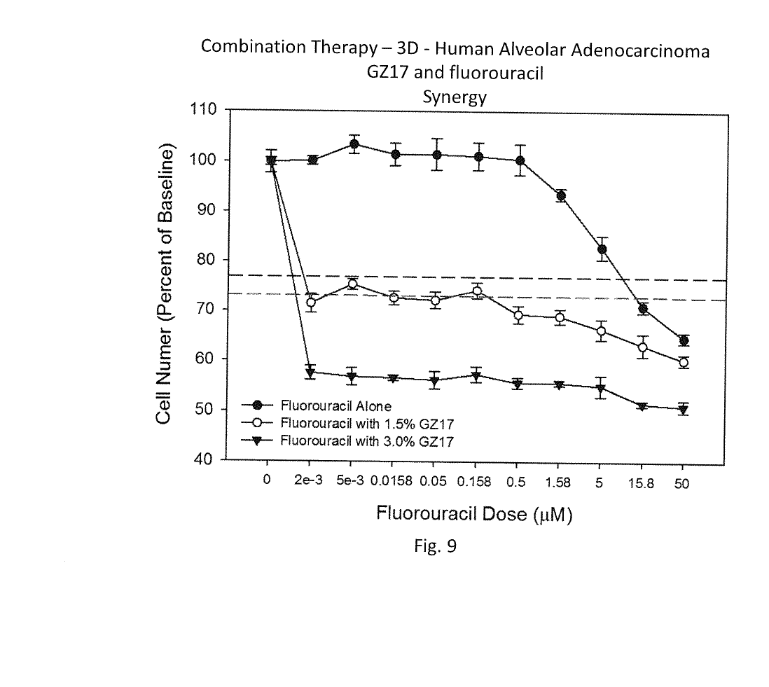

The study was again repeating using fluorouracil. Fluorouracil is an anti-metabolite chemotherapy. It mimics pyrimidine and incorporates into the DNA and RNA blocking the ability of the cell to divide, and eventually ending in death of the cancer cells. Other drugs in this category include cytarabine and 6-azauracil. The chemical structure of fluorouracil is shown in FIG. 7. The results are shown in FIGS. 8-9. The effect of GZ17 or fluorouracil alone on adenocarcinoma lung cancer cell clusters is shown in FIG. 8. Fluorouracil has a significant decrease in cell numbers at 1.58 μM and higher doses. As shown in FIG. 9, with the addition of 1.5% GZ17, there was an additive effect of the two compounds even at the lowest dose of fluorouracil, because the decline in cell number was greater than fluorouracil alone and greater than GZ17 alone (the upper dashed line). With 3% GZ17, a dramatic and immediate effect of GZ17 on the fluorouracil dose/response curve is demonstrated at the lowest doses of fluorouracil and at all of the doses tested, which are statistically below the effect of fluorouracil alone and GZ17 alone (p<0.001).

In further detail, Fluorouracil alone had little effect on lung cancer spheroids, until a dose of 1.58 μM or greater was reached. But when 1.5% GZ17 was added to the fluorouracil, the two caused 28% cancer cell death and this response increased with greater doses of doxorubicin. With fluorouracil added to 3% GZ17, there was 42% cancer cell death at the lowest fluorouracil concentration. This was nearly twice the effect of GZ17 alone in a concentration range in which fluorouracil alone had no effect at all.

### Example 3

The human ovarian cancer cell line (A1847) was used to create miniature tumors by loading the individual cells in a micromold according to U.S. Pat. App. Pub. No. 2010/0233239. Cells were pipetted into the mold and allowed 2-3 days to form 3-D clusters, mimicking miniature tumors. Cell clusters were removed from the mold by washing and gentle pipetting. Cell clusters were placed in media containing 0.5% FBS. The clusters were dispensed into 96-well plates at a density of approximately 10-15 clusters per well. The cells were tested against doxorubicin, paclitaxel, or fluorouracil, alone or in combination with GZ17 using the same methods described in Example 1, above.

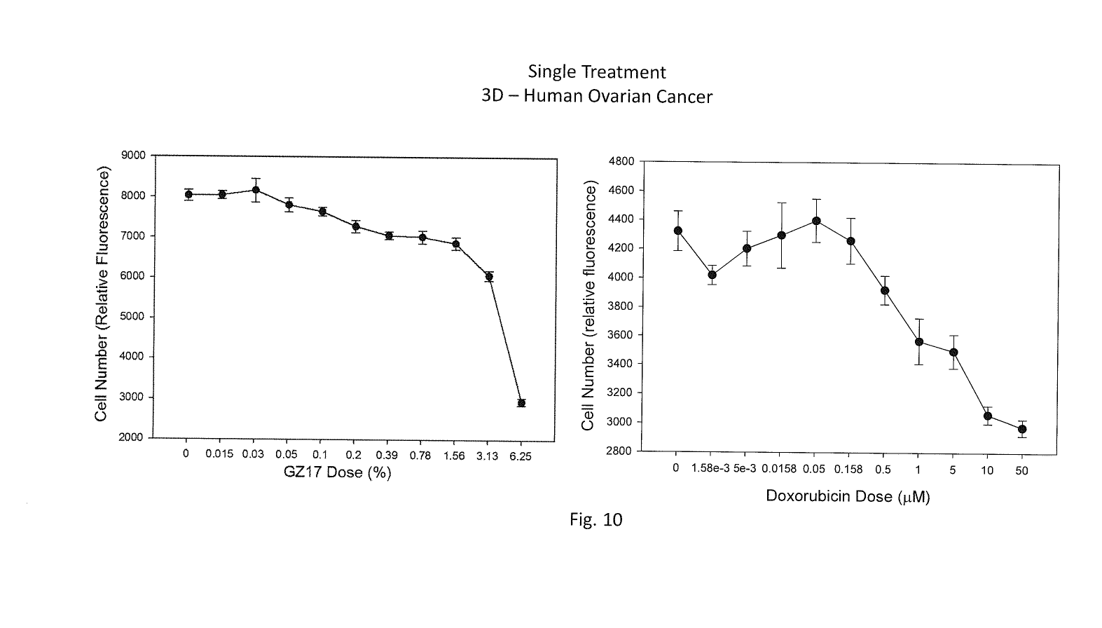

The results are shown in FIGS. 10-15. In FIG. 10, increasing doses of GZ17 decreased the number of live cells in the ovarian tumor cell clusters, statistically, starting at 0.39%. Doxorubicin dramatically reduced the number of live cells in the miniature tumor clusters. At 0.5 μM doxorubicin and all higher doses, there were statistically fewer live cells in the clusters than clusters that were not exposed to the drug (0 point on the X axis).

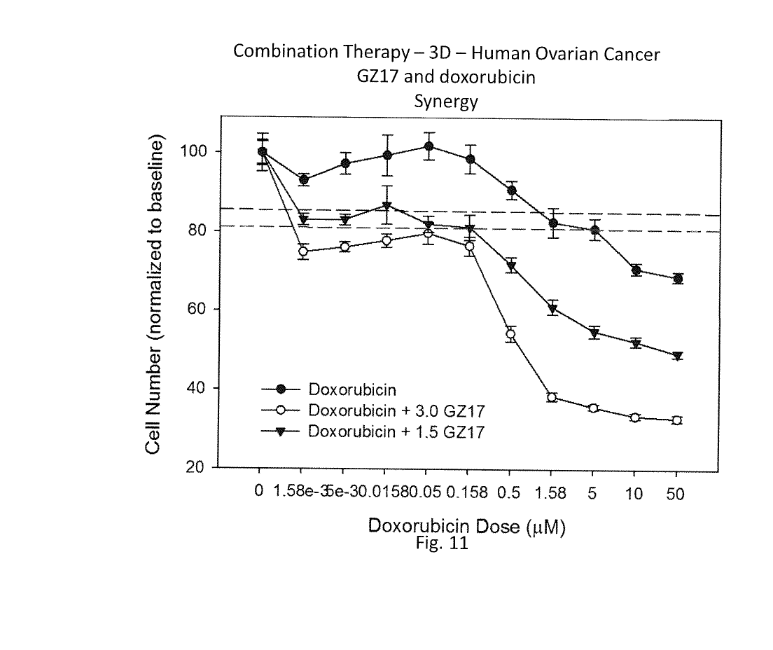

As shown unexpectedly in FIG. 11, when GZ17 was added to the doxorubicin, it increased the cell death at the lowest doxorubicin doses to levels slightly greater than the cell death measured by GZ17 alone (upper dashed line for 1.5% GZ17 and lower dashed line for 3.0% GZ17). The effects of doxorubicin, doxorubicin+1.5 GZ17 and doxorubicin+3.0 GZ17 are statistically different at every dose tested (p<0.05), except at 0.0158, 0.5 and 0.158 μM doxorubicin. However at 0.5 mM doxorubicin, the combination of either 1.5 or 3.0% GZ17 greatly increased the tumor cell death rate. This Figure, along with the Figures in Example 1 show that GZ17 in combination with doxorubicin both have a synergistic effect on lung cancer clusters and ovarian cancer clusters beyond their individual additive anticancer effects.

In further detail, doxorubicin alone at 1.58 mM reduced the viable ovarian cancer cells by 17%. 3% GZ17 alone caused 19% reduction in cancer cells. But the two added together caused a 62% decline in the ovarian cancer cells, much greater than either of the 2 anti-cancer agents alone. At 0.5 mM, a dose typically used in research to test the effects of doxorubicin, the two combined agents had a 3.5 times greater effect than 3% GZ17 alone, and a 7 times greater effect than doxorubicin alone.

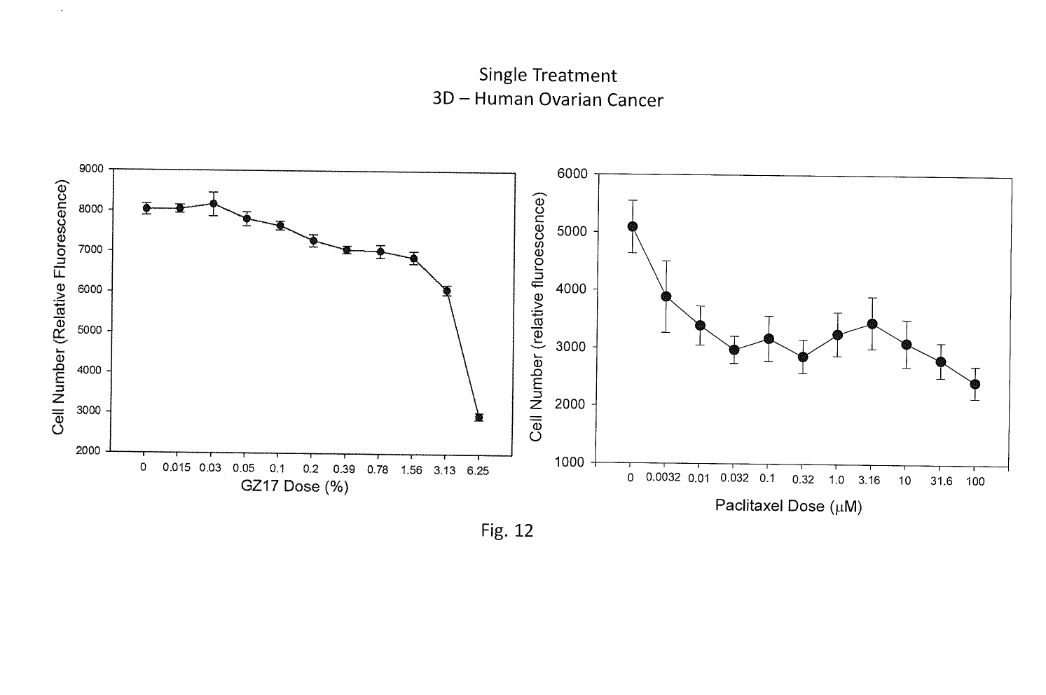

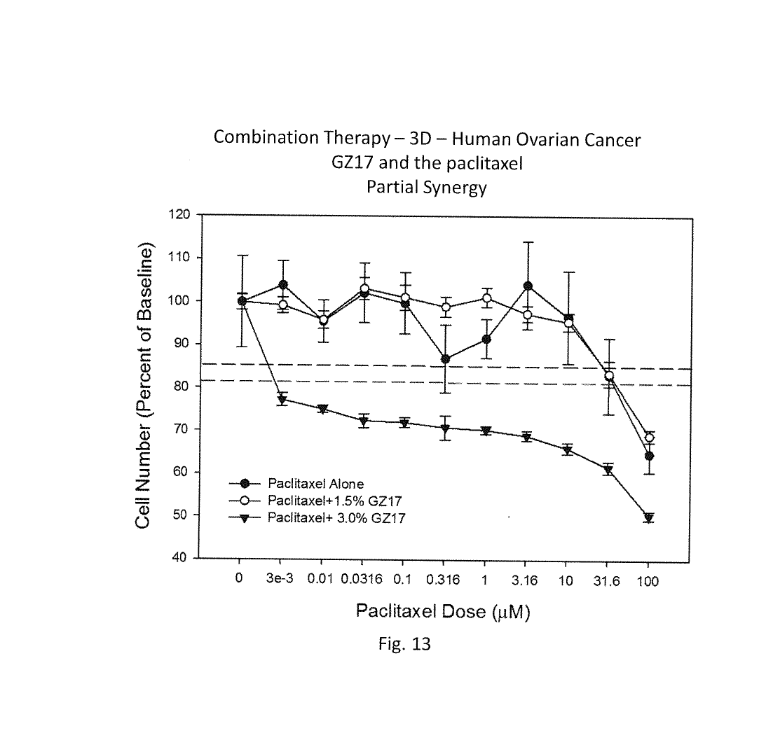

FIG. 12 shows that paclitaxel had an effect that was statistically significant at the 0.01 μM dose. As shown in FIG. 13, when paclitaxel and 1.5% GZ17 were added, there was no added or synergistic effect of the two compounds. However, at 3.0% GZ17 plus all of the doses of paclitaxel, there was a statistically significant (p<0.001) decline in the number of remaining live cells at all doses of paclitaxel. Thus, in ovarian cancer, there is a synergy between GZ17 and paclitaxel, but only at the higher GZ17 dose.

In further detail, paclitaxel alone had no effect on ovarian cancer until a dose of 31.6 μM or greater. However, when 3% GZ17 was added to paclitaxel, even at its lowest dose of 0.003, the effect was greater than either alone. At 3.16 μM paclitaxel, there was no effect on cancer cell death, but that same dose plus 3% GZ17 caused a 30% decline in viable cancer cell numbers. At 3% GZ17 and 10 mM paclitaxel, the additive effect of the 2 agents was 7 times greater than paclitaxel alone, and nearly 5 times greater than GZ17 alone.

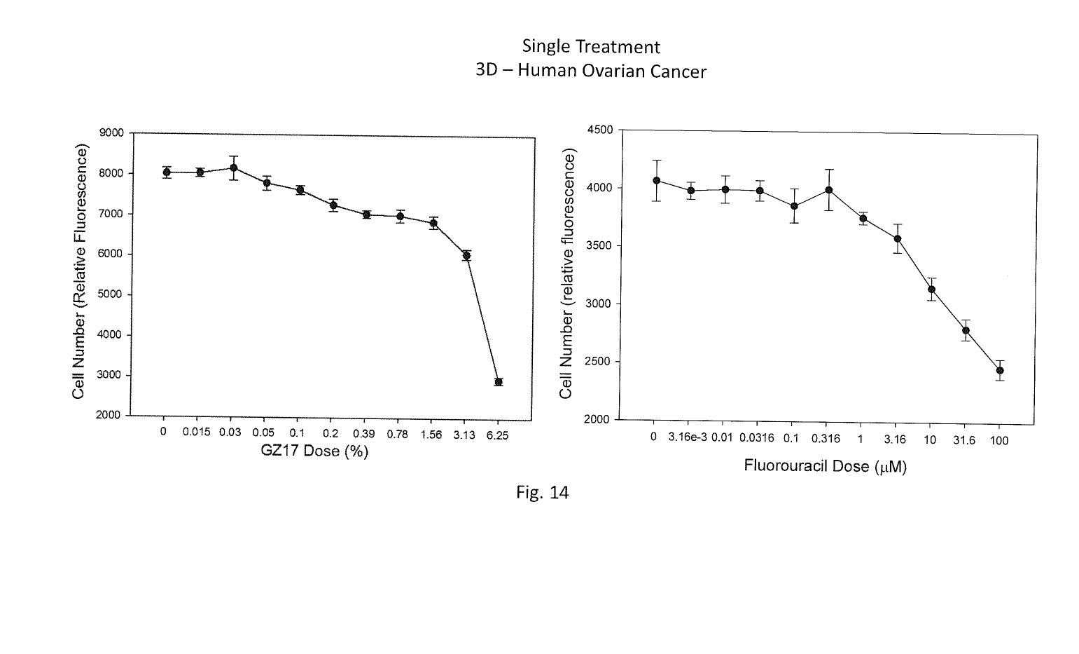

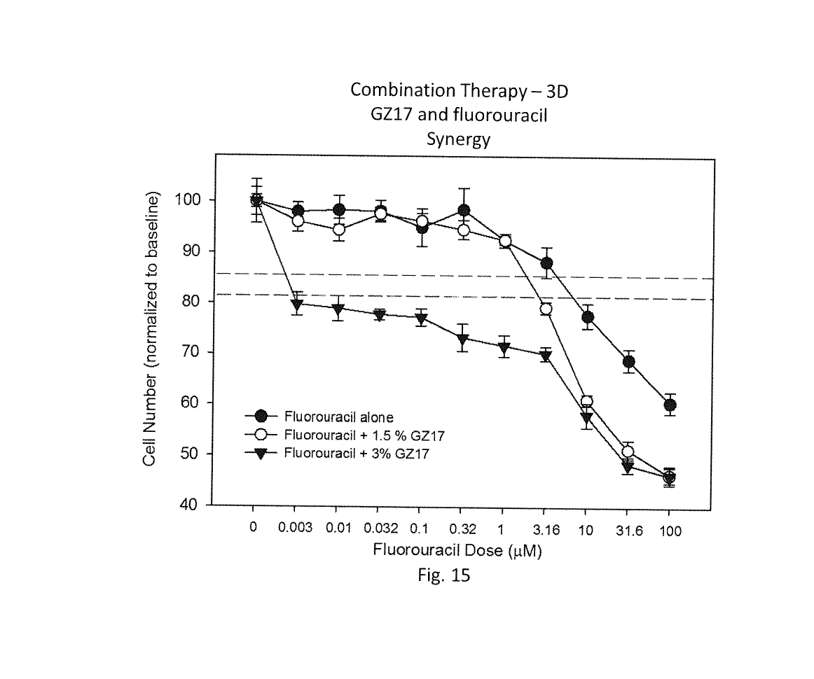

The effects of GZ17 or fluorouracil alone on ovarian cancer cell clusters are shown in FIG. 14. Fluorouracil achieved a significant decrease in cell numbers at 3.16 μM and higher doses (p<0.001). FIG. 15 shows that when fluorouracil was administered alone (black circles), the dose response was minimal. However, with the addition of 1.5% GZ17, there was an additive effect of the two compounds at the higher doses of fluorouracil (3.16 μM fluorouracil and higher), and greater than 1.5% GZ17 alone (upper dashed line). With 3% GZ17, there was a dramatic and immediate effect of GZ17 on the fluorouracil dose/response curve at the lowest doses of fluorouracil and at all of subsequent doses tested, with all doses tested greater than the effect of either compound alone, demonstrating a synergy between the two agents.

In further detail, fluorouracil alone had little effect on ovarian cancer spheroids, until a dose of 3.16 μM or greater was reached. But when 3% GZ17 was added to the fluorouracil, the combination was more effective. With 1 μM fluorouracil plus 3% GZ17, the response was 4.5 times greater than fluorouracil alone and 1.5 times greater than GZ17 alone.

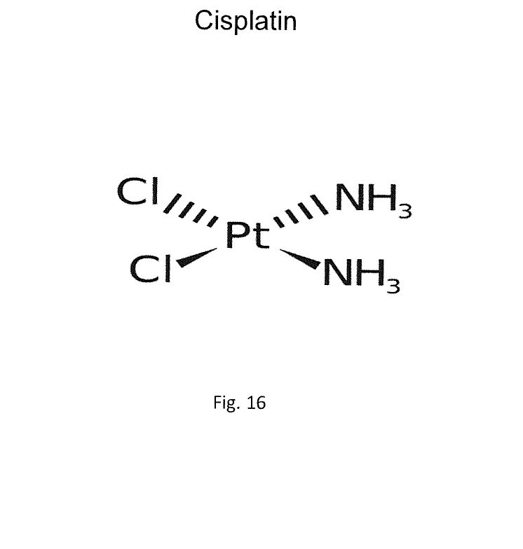

The same studies from Examples 1-2 were repeated, but the previously-tested chemotherapies were replaced by cisplatin. The chemotherapy classification of cisplatin (also known as platinol) is a platinum-containing that acts as an alkylating-like agent. By attaching to DNA, cisplatin blocks the DNA from uncoiling during cell division. The chemical structure of cisplatin is shown in FIG. 16. Other drugs that work like cisplatin are carboplatin, oxaliplatin, and nedaplatin. Other alkylating agents include chlorambucil, bendamustine, and melphalan.

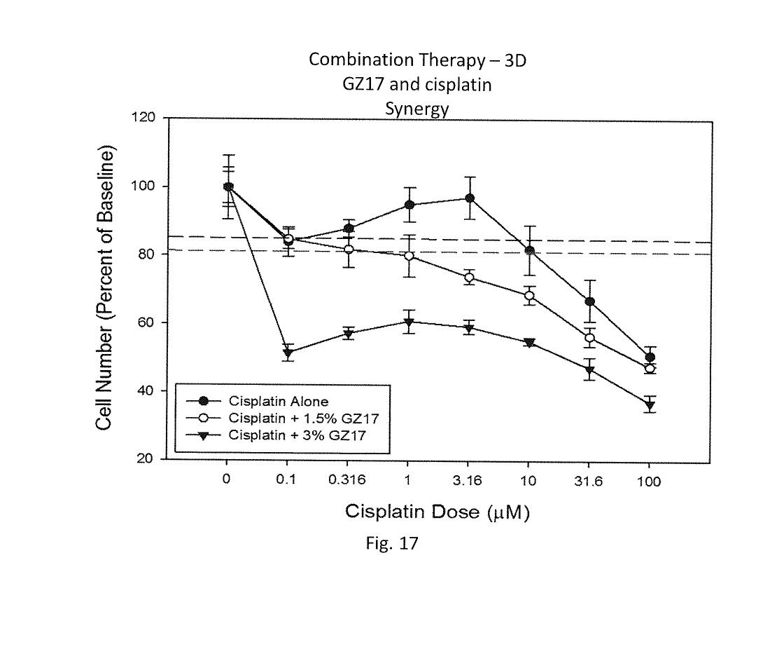

Cisplatin (also known as platinol) is a platinum-based alkylating agent. The effects of cisplatin alone on ovarian cancer cell clusters are shown in FIG. 17 (black circles). Cisplatin achieved a significant decrease in cell numbers only at the high dose of 31.6 μM and higher doses (p<0.05). However, with the addition of 1.5% GZ17, there was an additive effect of the two compounds at 0.316 μM cisplatin and higher). With 3% GZ17, there was a dramatic and immediate effect of GZ17 on the cisplatin dose/response curve at the lowest doses of cisplatin and at all of subsequent doses tested, demonstrating a synergy between the two agents.

In further detail, cisplatin alone had little effect on ovarian cancer spheroids until doses of 10 μM or greater. However, the addition of 3% GZ17 to cisplatin had an immediate effect. At the lowest dose of cisplatin (0.1 mM) the combination of cisplatin and 3% GZ17 killed 8 times more cancer cells than cisplatin alone at that same concentration and 3 times more cancer cells than GZ17 alone.

### Example 4

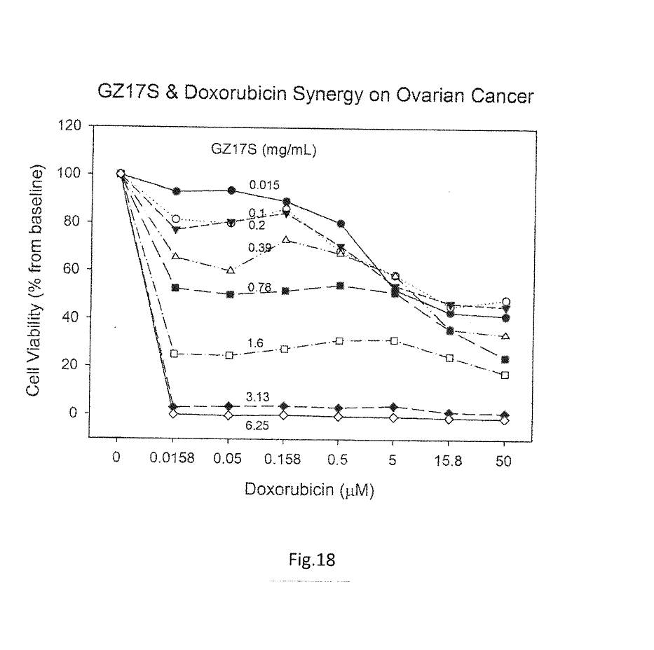

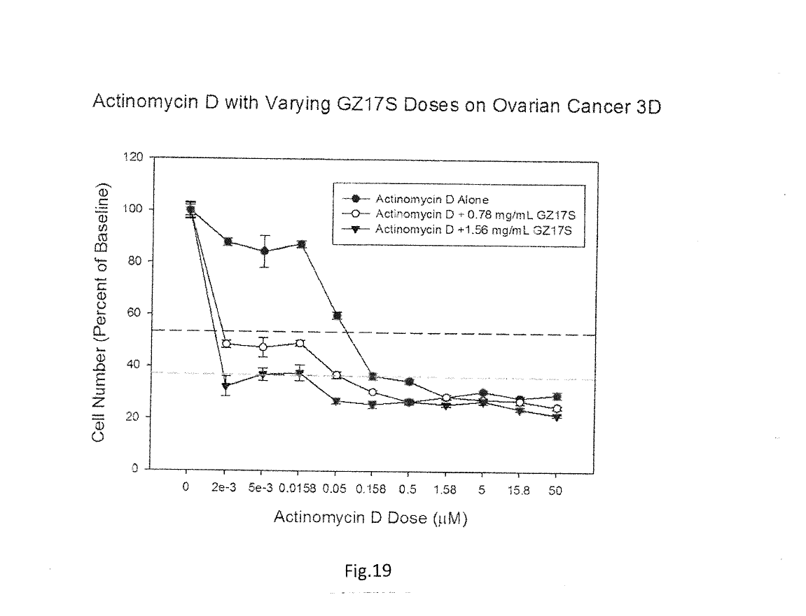

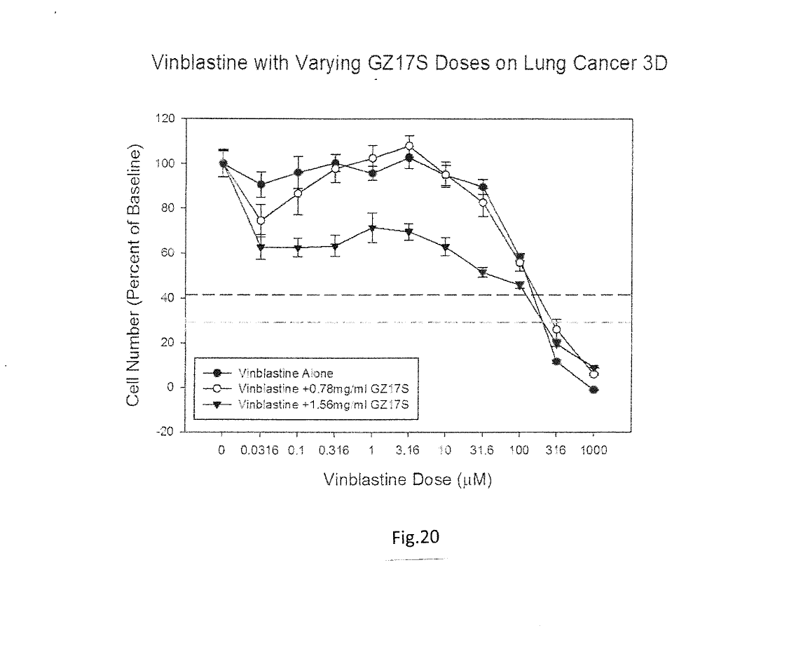

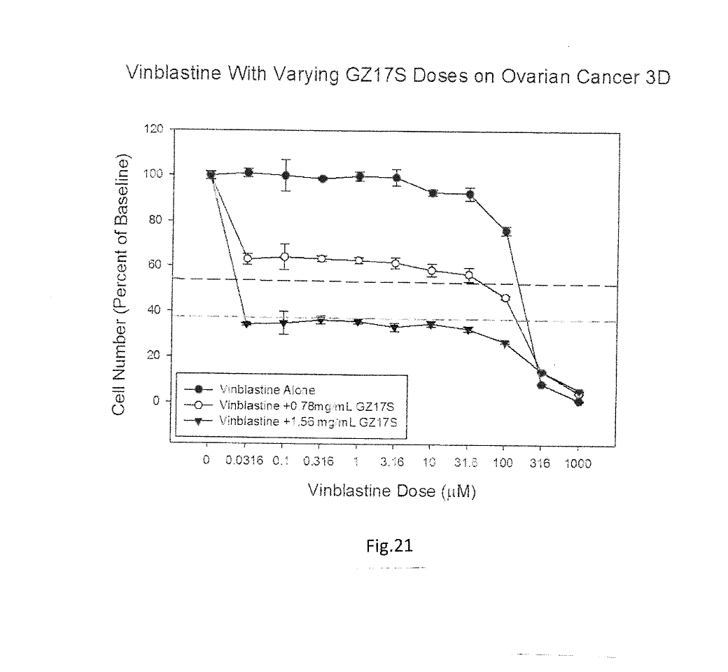

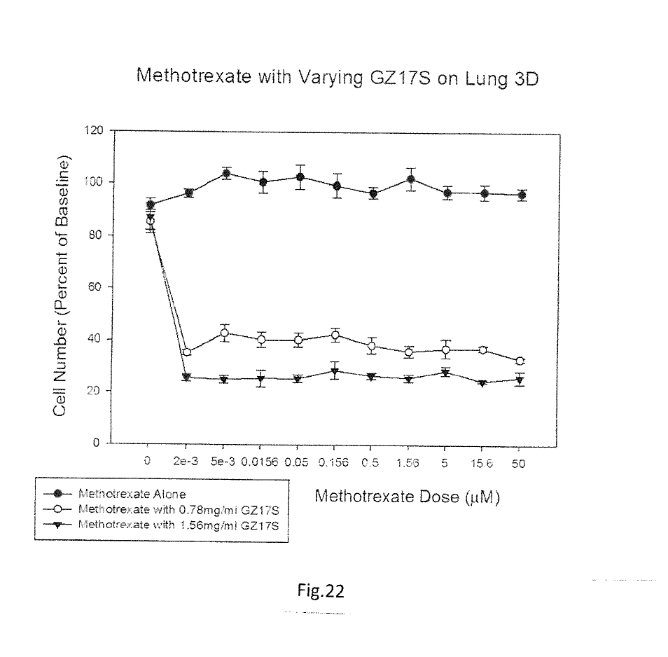

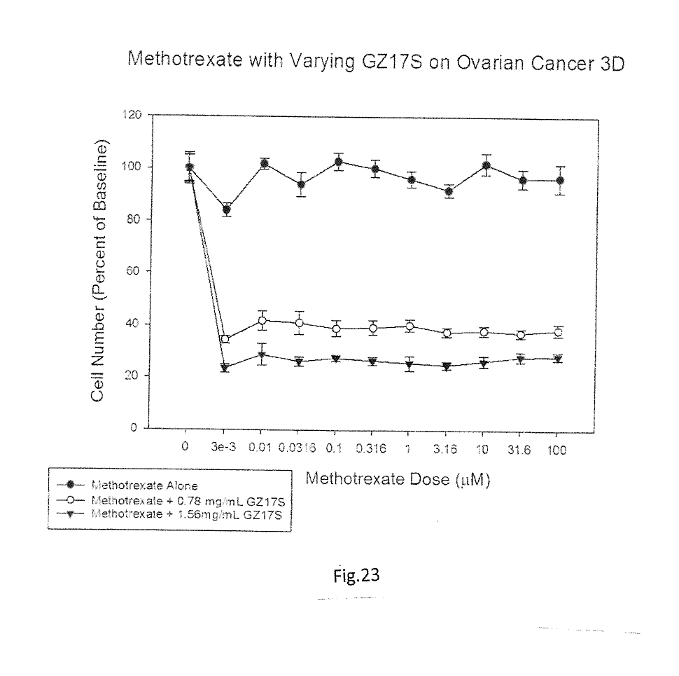

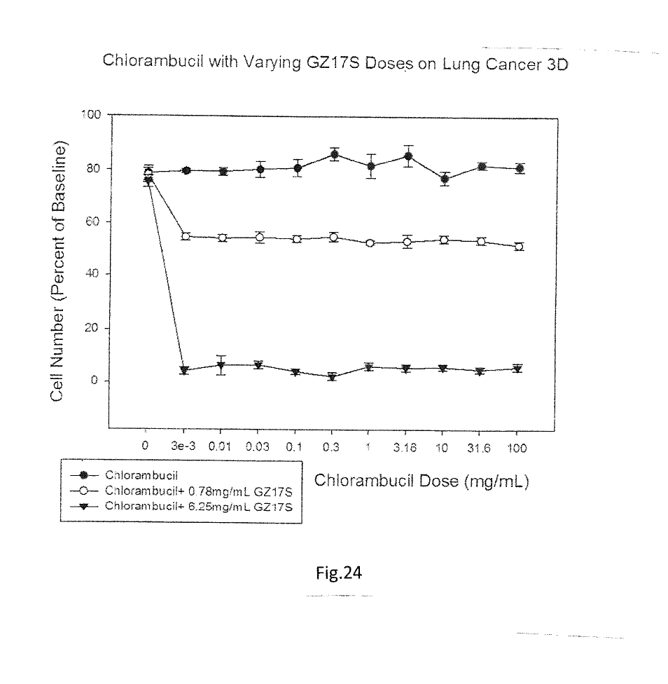

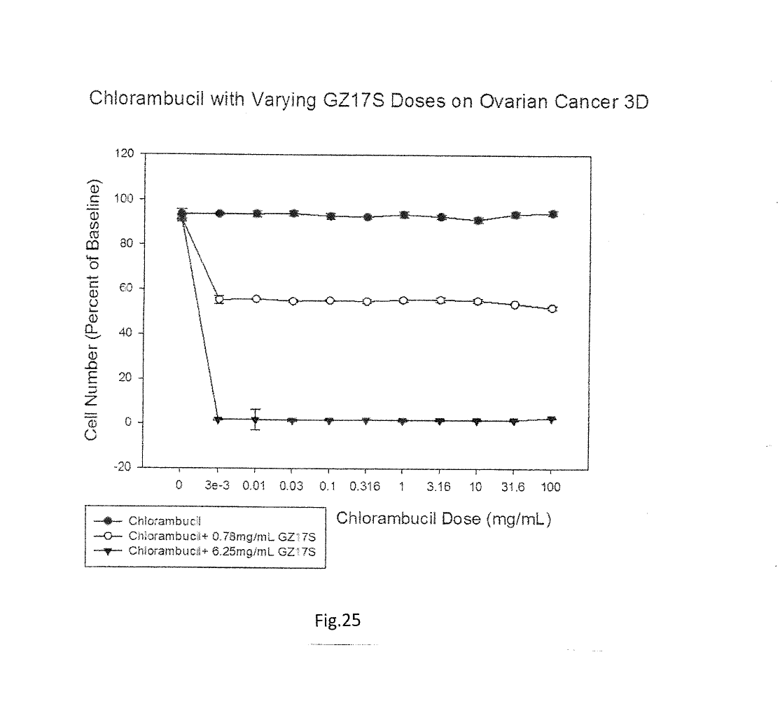

In this Example, a number of different chemotherapeutics were tested in combination with GZ17S, which is the solid form of the fortified aqueous decoction described in U.S. Pat. No. 8,039,025, prepared using a fortified decoction or tea as previously described which was then dried by lypholization to provide a solid (powder) product. The powder was reconstituted in deionized water and agitated to ensure substantial homogeneity. The reconstituted GZ17S product was tested, using the method of Examples 1-3, together with doxorubicin on ovarian cancer cells (FIG. 18); atenomycin D on ovarian cancer cells (FIG. 19), vinblastine on lung cancer cells (FIG. 20); vinblastine on ovarian cancer cells (FIG. 21); methotrexate on lung cancer cells (FIG. 22); methotrexate on ovarian cancer cells (FIG. 23); chlorambucil on lung cancer cells (FIG. 24); and chlorambucil on ovarian cancer cells (FIG. 25).

FIG. 18 depicts the results of an important and involved experiment where eight different doses of GZ17S and eight different doses of doxorubicin were tested together, resulting in 64 different dose combinations which were tested in six replicates per dose. The FIG. 18 graph is an extension of the data illustrated in FIG. 3. In FIG. 18, the doxorubicin dose is shown increasing on the X axis, and each dose is tested with the addition of eight different doses of GZ17S, and the latter are marked on each line within the graph. Thus, the top graph line (filled circles) is each dose of doxorubicin plus 0.015 micromoles of GZ17S. The next line down (open circles) is each dose of doxorubicin plus 0.1 micromoles of GZ17S, and so forth. This data demonstrate that with each increasing dose of GZ17S, the effect of doxorubicin on ovarian cancer cells is enhanced, even at the lowest doses of doxorubicin.

FIG. 19 illustrates the synergistic effects of combinations of atenomycin D and GZ17S on ovarian cancer cells, at essentially all of the doses tested.

Vinblastine, like paclitaxel, works by inhibiting the separation of microtubes. This drug in combination with GZ17S indicated synergy at higher doses (about 100-1000 micromoles) on both lung cancer (FIG. 20) and ovarian cancer (FIG. 21) cells. Overall, the paclitaxel/GZ17 combination was more efficient at causing cell death than the vinblastine/GZ17 combination.

Methotrexate, like fluorouracil, is an anti-metabolite. However, fluorouracil mimics pyrimidine, while methotrexate serves as a folic acid antagonist. Thus, while methotrexate is incorporated into cellular DNA and RNA, it acts at another site as compared with fluorouracil. As illustrated in FIG. 22, the addition of GZ17S causes significant cell death, even at the lowest methotrexate dose. Similarly, the addition of GZ17S to methotrexate dramatically decreases ovarian cancer cell viability (FIG. 23).

Like cisplatin, chlorambucil is an alkylating agent, but of the subcategory of nitrogen-mustard alkylating agents. Chlorambucil alone had no effect on lung cancer spheroids; however, the addition of GZ17S caused a decline in cell cancer viability at both doses tested (FIG. 24), a decline that is equivalent to that obtained by GZ17 alone. Similar results were obtained with ovarian cancer spheroids (FIG. 25).

In FIGS. 19, 20, and 21, the upper dashed lines indicate the percentage of cells still alive with a single dose of 1.5% GZ17S alone, whereas the lower dashed lines indicate the percentage of cells that are still alive with a single dose of 3.0% GZ17S alone.

### Discussion

As is evident from the foregoing Examples, the synergies between GZ17 and using drugs from the 4 traditional chemotherapy classes has been established. Additional chemotherapies include hormones (prednisone and dexamethasone), and monoclonal antibodies (rituximab and cetuximab). The other classes, hormones and antibodies cannot be tested in an in vitro setting. They require an intact immune system in order to have their effect.

It will be appreciated that the extent of synergy using GZ17 in combination with different chemotherapy classes can vary significantly, based upon the type of cancer cells being tested, the specific chemotherapy used, and the dosage levels employed. Thus, GZ17 may exhibit no significant synergy with a particular chemotherapeutic at a particular dosage level on one cell type, while giving dramatic synergy results with different cell types, specific chemotherapy drugs, and/or dosage levels. However, determining appropriate and useful synergies is well within the skill of the art, using the foregoing results as a guide.

For example, the intercalating antibiotics showed mixed results with doxorubicin as very synergistic with GZ17 for both lung and ovarian cancers, while actinomycin D was not. While they both intercalate DNA, they also have other actions that are unique to each drug and may explain why only doxorubicin showed synergy with GZ17. Thus, doxorubicin also inhibits topoisomerase II, preventing the relaxing of supercoiled DNA and thus blocking transcription. Further their binding sites to DNA are unique.

Both paclitaxel and vinblastine in combination with GZ17 had no synergy on lung cancer spheroids. But paclitaxel did show synergistic effects on ovarian cancer with 3% GZ17. Fluorouracil had strong synergy with GZ17, especially at 3% GZ17. But methotrexate had no effect alone on lung cancer spheroids and any effect of combining methotrexate and GZ17 was from the GZ17 alone. The same results were true in ovarian cancer with fluorouracil and GZ17 showing strong synergy, but no synergy of GZ17 plus methotrexate. Chlorambucil is also an alkylating agent, but of the subcategory of nitrogen-mustard alkylating agents. Cisplatin showed synergistic effects on ovarian cancer with GZ17, but another drug that had no effect on its own, chlorambucil, had no synergy with GZ17. In summary, chemotherapies that had no effect on the cancer spheroids on their own, exhibited no synergy with GZ17. But chemotherapeutic agents that demonstrated the ability to kill cancer cells on their own, tended to work in a synergistic manner with GZ17.

### Example 5

Many people drink tea made from the Arum palaestinum plant. LifePlus has supplied the fortified version of the tea as an energy drink and monitored the self-reported outcomes. Since 2012, nine individuals with cancer began taking GZ17 as an energy drink, supplied by LifePlus. Three of those people taking GZ17 who had cancer are now in complete remission. The other six people taking GZ17 self-reported a reduction in the tumor size. When quantified, the shrinkage in tumor size ranged from 25 to 50%. Of those patients ingesting GZ17, seven or 78% were administered chemotherapy at the same time that they were taking GZ17. For those people receiving chemotherapy while regularly drinking the GZ17 tea, 29% are in full remission. The remaining people have all had a positive response to their traditional chemotherapy, even when told by physicians that their life expectancy was initially poor.

The types of cancers people reported have included: Basil cell carcinoma, Melanoma, Lymphoma, Colon cancer, Metastasized lung cancer, Renal cell carcinoma, and Metastasized liver cancer. Chemotherapies that people were taking while drinking the GZ17 energy drink included docetaxel (taxatere), gemcitabine (gemzar), imatinib (gleevec) and camptosar. Docetaxel is an anti-mitotic chemotherapy of the same classification as paclitaxel. Gemcitabine is an anti-metabolite classification of chemotherapy in the same group as fluorouracil. Imatinib is a relatively new chemotherapy inhibiting tyrosine kinase activity and camptosar targets DNA topoisomerase 1, leading to DNA breakage. Thus of the known drugs being taken at the same time that these individuals were ingesting GZ17, one was in the same classification as fluorouracil and another was in the same category as paclitaxel.

The invention is applicable in the treatment of virtually all cancers, such as the following: Acute Lymphoblastic Leukemia, Adult; Acute Lymphoblastic Leukemia, Childhood; Acute Myeloid Leukemia, Adult; Acute Myeloid Leukemia, Childhood; Adrenocortical Carcinoma; Adrenocortical Carcinoma, Childhood; Adolescents, Cancer in; AIDS-Related Cancers; AIDS-Related Lymphoma; Anal Cancer; Appendix Cancer; Astrocytomas, Childhood; Atypical Teratoid/Rhabdoid Tumor, Childhood, Central Nervous System; Basal Cell Carcinoma; Bile Duct Cancer, Extrahepatic; Bladder Cancer; Bladder Cancer, Childhood; Bone Cancer, Osteosarcoma and Malignant Fibrous Histiocytoma; Brain Stem Glioma, Childhood; Brain Tumor, Adult; Brain Tumor, Brain Stem Glioma, Childhood; Brain Tumor, Central Nervous System Atypical Teratoid/Rhabdoid Tumor, Childhood; Brain Tumor, Central Nervous System Embryonal Tumors, Childhood; Brain Tumor, Astrocytomas, Childhood; Brain Tumor, Craniopharyngioma, Childhood; Brain Tumor, Ependymoblastoma, Childhood; Brain Tumor, Ependymoma, Childhood; Brain Tumor, Medulloblastoma, Childhood; Brain Tumor, Medulloepithelioma, Childhood; Brain Tumor, Pineal Parenchymal Tumors of Intermediate Differentiation, Childhood; Brain Tumor, Supratentorial Primitive Neuroectodermal Tumors and Pineoblastoma, Childhood; Brain and Spinal Cord Tumors, Childhood (Other); Breast Cancer ; Breast Cancer and Pregnancy; Breast Cancer, Childhood; Breast Cancer, Male; Bronchial Tumors, Childhood; Burkitt Lymphoma; Carcinoid Tumor, Childhood; Carcinoid Tumor, Gastrointestinal; Carcinoma of Unknown Primary; Central Nervous System Atypical Teratoid/Rhabdoid Tumor, Childhood; Central Nervous System Embryonal Tumors, Childhood; Central Nervous System (CNS) Lymphoma, Primary; Cervical Cancer; Cervical Cancer, Childhood; Childhood Cancers; Chordoma, Childhood; Chronic Lymphocytic Leukemia; Chronic Myelogenous Leukemia; Chronic Myeloproliferative Disorders; Colon Cancer; Colorectal Cancer, Childhood; Craniopharyngioma, Childhood; Cutaneous T-Cell Lymphoma; Embryonal Tumors, Central Nervous System, Childhood; Endometrial Cancer; Ependymoblastoma, Childhood; Ependymoma, Childhood; Esophageal Cancer; Esophageal Cancer, Childhood; Esthesioneuroblastoma, Childhood; Ewing Sarcoma Family of Tumors; Extracranial Germ Cell Tumor, Childhood; Extragonadal Gem). Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Gastrointestinal Stromal Tumor (GIST); Gastrointestinal Stromal Cell Tumor, Childhood; Germ Cell Tumor, Extracranial, Childhood; Germ Cell Tumor, Extragonadal; Germ Cell Tumor, Ovarian; Gestational Trophoblastic Tumor; Glioma, Adult; Glioma, Childhood Brain Stem; Hairy Cell Leukemia; Head and Neck Cancer; Heart Cancer, Childhood; Hepatocellular (Liver) Cancer, Adult (Primary); Hepatocellular (Liver) Cancer, Childhood (Primary); Histiocytosis, Langerhans Cell; Hodgkin Lymphoma, Adult; Hodgkin Lymphoma, Childhood; Hypopharyngeal Cancer; Intraocular Melanoma; Islet Cell Tumors (Endocrine Pancreas); Kaposi Sarcoma; Kidney (Renal Cell) Cancer; Kidney Cancer, Childhood; Langerhans Cell Histiocytosis; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoma, AIDS-Related; Lymphoma, Burkitt; Lymphoma, Cutaneous T-Cell; Lymphoma, Hodgkin, Adult; Lymphoma, Hodgkin, Childhood; Lymphoma, Non-Hodgkin, Adult; Lymphoma, Non-Hodgkin, Childhood; Lymphoma, Primary Central Nervous System (CNS); Macroglobulinemia, Waldenström; Malignant Fibrous Histiocytoma of Bone and Osteosarcoma; Medulloblastoma, Childhood; Medulloepithelioma, Childhood; Melanoma; Melanoma, Intraocular (Eye); Merkel Cell Carcinoma; Mesothelioma, Adult Malignant; Mesothelioma, Childhood; Metastatic Squamous Neck Cancer with Occult Primary; Mouth Cancer; Multiple Endocrine Neoplasia Syndromes, Childhood; Multiple Myeloma/Plasma Cell Neoplasm; Mycosis Fungoides; Myelodysplastic Syndromes; Myelodysplastic/Myeloproliferative Neoplasms; Myelogenous Leukemia, Chronic; Myeloid Leukemia, Adult Acute; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin Lymphoma, Adult; Non-Hodgkin Lymphoma, Childhood; Non-Small Cell Lung Cancer; Oral Cancer, Childhood; Oral Cavity Cancer, Lip and; Oropharyngeal Cancer; Osteosarcoma and Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell Tumors; Papillomatosis, Childhood; Paranasal Sinus and Nasal Cavity Cancer; Parathyroid Cancer; Penile Cancer; Pharyngeal Cancer; Pineal Parenchymal Tumors of Intermediate Differentiation, Childhood; Pineoblastoma and Supratentorial Primitive Neuroectodermal Tumors, Childhood; Pituitary Tumor; Plasma Cell Neoplasm/Multiple Myeloma; Pleuropulmonary Blastoma, Childhood; Pregnancy and Breast Cancer; Primary Central Nervous System (CNS) Lymphoma; Prostate Cancer; Rectal Cancer; Renal Cell (Kidney) Cancer; Renal Pelvis and Ureter, Transitional Cell Cancer; Respiratory Tract Cancer with Chromosome 15 Changes; Retinoblastoma; Rhabdomyosarcoma, Childhood; Salivary Gland Cancer; Salivary Gland Cancer, Childhood; Sarcoma, Ewing Sarcoma Family of Tumors; Sarcoma, Kaposi; Sarcoma, Soft Tissue, Adult; Sarcoma, Soft Tissue, Childhood; Sarcoma, Uterine; Sézary Syndrome; Skin Cancer (Nonmelanoma); Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Cell Carcinoma; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Testicular Cancer, Childhood; Throat Cancer; Thymoma and Thymic Carcinoma; Thymoma and Thymic Carcinoma, Childhood; Thyroid Cancer; Thyroid Cancer, Childhood; Transitional Cell Cancer of the Renal Pelvis and Ureter; Trophoblastic Tumor, Gestational; Unknown Primary Site, Carcinoma of, Adult; Unknown Primary Site, Cancer of, Childhood; Unusual Cancers of Childhood; Ureter and Renal Pelvis, Transitional Cell Cancer; Urethral Cancer; Uterine Cancer, Endometrial; Uterine Sarcoma; Vaginal Cancer; Vaginal Cancer, Childhood; Vulvar Cancer; Waldenstrom Macroglobulinemia; Wilms Tumor; Women's Cancers.

We claim:

1. A therapeutic anticancer combination comprising coordinated, preselected therapeutically synergistic amounts of at least one chemotherapeutic agent and a fortified decoction dosage form, wherein said fortified decoction dosage form comprises from about 10 mg to about 6,000 mg each of β-sitosterol, isovanillin, and linolenic acid, such that the combination has a therapeutic effect on cancerous tissue which is greater than the sum of the individual therapeutic effects of the fortified decoction dosage form and the at least one chemotherapeutic agent on the cancerous tissue.

2. The therapeutic anticancer combination of claim 1, wherein said combination consists of said chemotherapeutic agent and said fortified decoction dosage form.

3. The therapeutic anticancer combination of claim 1, wherein said fortified decoction dosage form is in the form of an aqueous dispersion, gel, ampule, powder, capsule, pill, tablet, or combination thereof.

4. The therapeutic anticancer combination of claim 1, wherein said fortified decoction dosage form comprises plant extract of the genus Arum, fortified with effective amounts of β-sitosterol, isovanillin, and linolenic acid not derived from said plant extract.

5. The therapeutic anticancer combination of claim 1, wherein said fortified decoction dosage form consists essentially of plant extract of the genus Arum, fortified with effective amounts of β-sitosterol, isovanillin, and linolenic acid not derived from said plant extract, optionally dispersed or dissolved in an aqueous solvent system.

6. The therapeutic anticancer combination of claim 1, wherein said chemotherapeutic agent is selected from the group consisting of antimetabolites, anthracyclines, mitotic inhibitors, alkylating agents, and combinations thereof.

7. The therapeutic anticancer combination of claim 1, said chemotherapeutic agent having an anticancer effect when administered alone and without the presence of said fortified decoction dosage form.

8. A method of treating human cancer, comprising administering the therapeutic anticancer combination of claim 1 to a human cancer patient.

9. The method of claim 8, wherein said fortified decoction dosage form is administered on a daily basis.

10. The method of claim 8, wherein said administering comprises administering as a single unit dosage a composition comprising a therapeutically effective amount of said fortified decoction dosage form and a therapeutically effective amount of said chemotherapeutic agent.

11. The method of claim 8, wherein said administering comprises co-administering a therapeutically effective amount of said fortified decoction dosage form and a therapeutically effective amount of said chemotherapeutic agent.

12. The method of claim 8, wherein said subject is undergoing chemotherapy treatment with said chemotherapeutic agent prior to said administering of said fortified decoction dosage form.

13. The method of claim 8, wherein said fortified decoction dosage form in combination with said chemotherapeutic agent achieves a therapeutic synergy in treating said cancer in said subject.

14. The method of claim 8, wherein the cancer of said subject is improved as compared to a subject treated with said chemotherapeutic agent not in combination with said fortified decoction dosage form.
